(19) United States
(12) Patent Application Publication
(10) Pub. No.: US 2020/0298172 A1
(43) Pub. Date: Sep. 24, 2020

(54) METHODS OF PURIFYING INDUSTRIAL GAS STREAMS

(71) Applicant: CHEVRON U.S.A. INC., San Ramon, CA (US)

(72) Inventors: Marcus Dutra E. MELLO, Moraga, CA (US); Raja JADHAV, Benecia, CA (US); Zunqing Alice HE, San Rafael, CA (US); Joshua A. THOMPSON, Martinez, CA (US)

(21) Appl. No.: 16/821,280

(22) Filed: Mar. 17, 2020

Related U.S. Application Data
(60) Provisional application No. 62/820,429, filed on Mar. 19, 2019.

(51) Int. Cl. B01D 53/14; B01D 53/26; B01D 15/36; B01D 15/20; C07C 29/94

(52) U.S. Cl. CPC B01D 53/1425; B01D 53/263; B01D 53/1493; B01D 2252/60; B01D 15/203; C07C 29/94; B01D 2252/2023; B01D 15/363

(57) ABSTRACT

Processes for removing a sulfide or a degradation product thereof from in a gas dehydration system are disclosed along with corresponding gas dehydration systems. The processes and systems include contacting a stream comprising the sulfide or a degradation product thereof with an anionic resin to remove at least a portion of the sulfide or a degradation product thereof from the stream. The processes and systems can also be used in the removal of mercury from gas dehydration systems.

### FIELD

This application is directed to the removal of a sulfur-containing additive or a degradation product thereof from a gas dehydration system using an anionic resin.

### BACKGROUND

Dehydration of natural gas using a glycol such as a triethylene glycol is a common process operation before natural gas can move from the wellhead into further processing or is used for sales. In some production wells, mercury is also present in natural gas and one of the proposed options to abate the mercury content is to react it with a sulfur-containing additive. The sulfur-containing additive reacts with the mercury and the resulting product needs to be removed from the gas dehydration system. If the sulfur-containing additive is not removed prior the glycol regeneration step, which is carried out at a temperature of about 200° C., the sulfur-containing additive can decompose. The decomposition products the sulfur-containing additive can precipitate or foul inside the regeneration vessel/heat exchanger and are thus undesirable. There is therefore a need in the art to remove the sulfur-containing additive or degradation products thereof from the gas regeneration system.

### SUMMARY

A process for removing a sulfur-containing additive or a degradation product thereof in a gas dehydration system is disclosed. The process comprises contacting a stream comprising the sulfur-containing additive or a degradation product thereof with an anionic resin to remove at least a portion of the sulfur-containing additive or the degradation product thereof from the stream. In some embodiments, the stream further comprises mercury and the anionic resin removes at least a portion of the mercury from the stream. The sulfur-containing additive can be selected from the group consisting of sulfides, hydrosulfides, inorganic and organic polysulfides, inorganic and organic thiocarbamates, mercaptans, thiourea, sulfur-containing polymers, sulfanes, and combinations thereof. In some embodiments, the process includes removing a sulfur-containing additive and the sulfur-containing additive comprises a sulfide selected from the group consisting of a polysulfide, a polysulfide salt, a sulfide salt, or a combination thereof, and can be, for example, sodium polysulfide. In some embodiments, the process includes removing a degradation product of the sulfur-containing additive, wherein the degradation product comprises a thiosulfate, a sulfite, a sulfate, elemental sulfur, hydrogen sulfide, or a combination thereof.

In some embodiments, the anionic resin can be contacted with an alkaline or chloride solution to regenerate the anionic resin after the anionic resin has been used to remove the sulfur-containing additive or the degradation product thereof. The resulting regenerated resin can be contacted with a stream comprising a sulfur-containing additive. In some embodiments, the gas dehydration system includes at least two anionic resin beds and wherein the stream can contact a first anionic resin bed while a second resin bed is regenerating. In some embodiments, the gas dehydration system includes an in-line anionic resin bed.

In some embodiments, the anionic resin has a sulfur capacity of at least 1 wt %, at least 2 wt %, at least 3 wt %, at least 4 wt %, at least 5 wt %, at least 6 wt %, or at least 7 wt %, or from 1 wt % to 14 wt %, or from 4 wt % to 14 wt %. The anionic resin can have a pore size of from 20 Angstroms to 120 Angstroms. In some embodiments, the anionic resin includes a chlorine or hydroxide anion. In some embodiments, the anionic resin includes a trimethylamine or trialkylbenzyl ammonium functional group. In some embodiments, an adsorbent selected from activated carbon, alumina, silica, zeolite, iron-oxide based adsorbent, or a supported metal is used in addition to the anionic resin to remove the sulfur-containing additive, the degradation product of a sulfur-containing additive, and/or the mercury from the stream.

The stream treated by the anionic resin can include water, a glycol, a sulfur-containing additive and/or a degradation product of a sulfur-containing additive, and mercury. In some embodiments, the stream can include at least 94 wt % glycol and can include no more than 98 wt % glycol. In some embodiments, the glycol comprises triethylene glycol. In some embodiments, the gas dehydration system includes a glycol regenerator, and the stream is contacted with the resin upstream of the glycol regenerator, downstream of the glycol regenerator, or a combination thereof. In some examples, the stream can be contacted with the resin upstream of the glycol regenerator and the stream can remove a least a portion of the sulfur-containing additive and at least a portion of the mercury from the stream. For example, the content of the sulfur-containing additive in the stream can be reduced by at least 50%, at least 60%, at least 70%, at least 80%, or at least 90%. In some examples, the stream can be contacted with the resin downstream of the glycol regenerator and the resin can remove at least a portion of the sulfur-containing additive and/or the degradation product of the sulfur-containing additive (and optionally mercury) from the stream. For example, the content of the sulfur-containing additive and/or the degradation product of the sulfur-containing additive in the stream can be reduced by at least 50%, at least 60%, at least 70%, at least 80%, or at least 90%.

In some embodiments, a natural gas rehydration system for removing a sulfide and mercury from a natural gas stream comprises a glycol contactor wherein natural gas combined with water and mercury is contacted with a glycol and wherein the glycol contactor includes a glycol feed that includes a sulfide for complexing mercury, said glycol contactor producing a rich glycol stream including the water, the sulfide, and the mercury and a natural gas stream; a glycol regenerator that receives the rich glycol stream from the glycol contactor and separates the water from the rich glycol stream to produce a water vapor stream and a lean glycol stream that is recycled as the glycol feed in the glycol contactor; and an anionic resin bed including an anionic resin provided upstream of the glycol regenerator, downstream of the glycol regenerator, or a combination thereof, for receiving a stream including the sulfide and/or a degradation product thereof and mercury, wherein the anionic resin bed removes at least a portion of the sulfide or the degradation product thereof and at least a portion of the mercury from the stream, wherein the sulfide is selected to the group consisting of sulfides, hydrosulfides, and inorganic and organic polysulfides. In some embodiments, the anionic resin bed can be an in-line resin bed. In some embodiments, the system can include at least two anionic resin beds such that a first anionic resin bed can be used to remove sulfide or the degradation product thereof and mercury from the stream while a second anionic resin bed can be regenerated using an alkaline or chloride solution. The anionic resin bed can be located upstream from glycol regenerator and a portion of the rich glycol stream can be fed to the anionic resin bed and/or the anionic resin bed can be located downstream from glycol regenerator and a portion of the lean glycol stream can be fed to the anionic resin bed. The portion of the rich glycol stream and/or lean glycol stream fed to the anionic resin bed can be 0.5-50% by volume or 0.5-2% by volume. In some embodiments, the additional sulfide can be added to the lean glycol stream prior to recycling the lean glycol stream to the glycol contactor.

The details of one or more embodiments are set forth in the descriptions below. Other features, objects, and advantages will be apparent from the description and from the claims.

### BRIEF DESCRIPTION OF THE FIGURES

FIG. 1 is a process flow diagram depicting a natural gas dehydration system that includes in line anionic resin beds for removing a sulfur-containing additive from the glycol streams.

FIG. 2 is a process flow diagram depicting a natural gas dehydration system that includes slip lines for diverting a portion of glycol streams to anionic resin beds to remove a sulfur-containing additive.

### DETAILED DESCRIPTION

Before the present methods and systems are disclosed and described, it is to be understood that the methods and systems are not limited to specific methods, specific components, or to particular systems. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. The term “exemplary” means “an example of” and is not intended to convey an indication of a preferred or ideal embodiment. “Such as” is not used in a restrictive sense, but for explanatory purposes.

The application describes a gas dehydration system for removing a sulfur-containing additive such as a sulfide or a degradation product of a sulfur-containing additive from a gas stream using an anionic resin. In particular, the gas dehydration system is a natural gas dehydration system that uses anionic resins for the removal of a sulfur-containing additive or a degradation product thereof used to remove mercury present in a glycol stream.

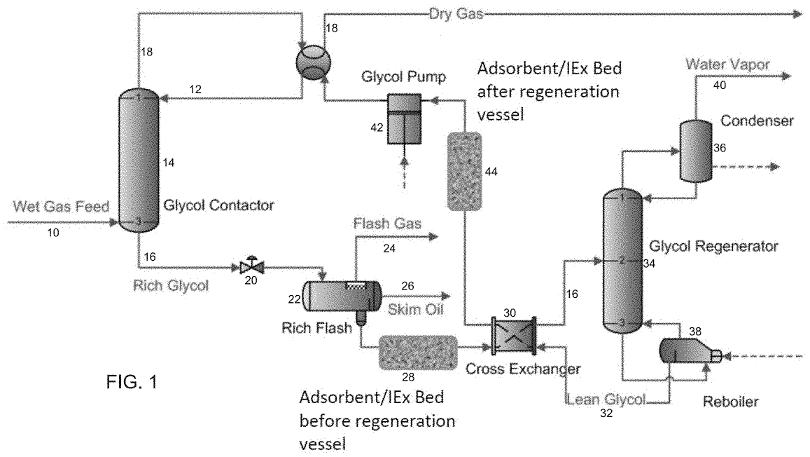

As illustrated in FIG. 1, a wet gas feed 10 and a glycol feed 12 are fed to a glycol contactor 14 and separated into a rich glycol stream 16 and a dry gas stream 18. The glycol contactor 14 is typically pressurized and includes tray columns or packed columns. The glycol feed 12 is fed at the top of the glycol contactor 14 and flows counter to the wet gas feed 10 fed to the bottom of the glycol contactor 14. The wet gas feed 10 is typically a natural gas feed that is includes water vapor and that can be saturated with water. The natural gas feed typically includes low molecular weight hydrocarbons such as methane, ethane, propane, and other paraffinic hydrocarbons that are typically gases at room temperature. The wet gas feed 10 can also include volatile mercury, including elemental mercury, in levels ranging from about 0.01 μg/Nm³ to 5000 μg/Nm³ (as measured according to ASTM D 6350). The glycol feed 12 includes glycol, which removes water from the wet gas feed 10 to produce the dry natural gas stream 18. In some embodiments, the glycol feed 12 includes triethylene glycol (TEG), tetraethylene glycol, or a combination thereof. In some embodiments, the glycol feed 12 includes TEG. In some embodiments, the glycol feed 12 includes 97-99.5 wt % glycol and 0.5-3 wt % water.

In order to remove mercury from the rich glycol stream 16, a sulfur-containing additive is included in the glycol feed 12. The sulfur-containing additive is capable of reacting with volatile mercury in natural gas after absorbing in the glycol solvent. The sulfur-containing additive can be selected from the group consisting of sulfides, hydrosulfides, inorganic and organic polysulfides, inorganic and organic thiocarbamates, mercaptans, thiourea, sulfur-containing polymers, sulfanes, and combinations thereof. In some embodiments, the sulfur-containing additive can be a sulfide selected from the group consisting of sulfides, hydrosulfides, inorganic polysulfides, organic polysulfides, or combinations thereof. Specific examples of sulfides include ammonium polysulfide, sodium polysulfide, potassium polysulfide, calcium polysulfide, sodium hydrosulfide, potassium hydrosulfide, ammonium hydrosulfide, sodium sulfide, potassium sulfide, calcium sulfide, magnesium sulfide, ammonium sulfide, and mixtures thereof.

The amount of sulfur-containing additives added to the glycol solution is determined by the effectiveness of the sulfur-containing additive employed. The amount is at least equal to the amount of mercury in the gas on a molar basis (1:1), if not in an excess amount. In some embodiments, the molar ratio ranges from 5:1 to 10,000:1, from 10:1 to 5000:1, or from 50:1 to 2500:1. In some embodiments, a sufficient amount of the sulfur-containing additive is added to the glycol contactor 14 for a sulfur-containing additive concentration, and specifically a sulfide concentration, ranging from 0.01 M to 10M, from 0.1M to 5M, from 0.3M to 4M, or from 0.5M to 4M. If the sulfur-containing additive is an organic or inorganic polysulfide, sulfane or mercaptan, the moles of sulfur-containing additive are calculated on the same basis as the amount of sulfur present. In some embodiments, the sulfur-containing additive is a sulfide and the amount of sulfide used is up to 15,000 ppm (expressed as total sulfur).

The rich glycol stream 16 leaving the glycol contactor 10 includes the sulfur-containing additive and the mercury, along with water from the wet gas feed 10 and glycol. In some embodiments, the glycol present in the rich glycol stream 16 is present in an amount of from 94% by weight to 98% by weight. The rich glycol stream 16 is depressurized by flashing the pressurized liquid through an expansion valve 20. The rich glycol stream 16 is then run to rich flash tank 22 wherein the rich glycol stream has flash gas 24 and skim oil 26 removed from the rich glycol stream 16.

As shown in FIG. 1, the gas dehydration system can optionally include an anionic resin bed 28 and the rich glycol stream 16 leaving the rich flash tank 22 can be fed to the anionic resin bed. The anionic resin bed 28 includes an anionic resin, which contacts the rich glycol stream 16 and reacts with the sulfur-containing additive to remove at least a portion of the sulfur-containing additive from the rich glycol stream 16. In some embodiments, the anionic resin bed reduces the content of the sulfur-containing additive, e.g., the sulfide content, in the rich glycol stream is reduced by at least 10%, at least 20%, at least 30%, at least 40%, at least 50%, at least 60%, at least 70%, at least 80%, or at least 90% by weight.

The anionic resin is selected to absorb or react with the sulfur-containing additive and can also be used to absorb or react with mercury present in the rich glycol stream 16. In some embodiments, the anionic resin has a sulfur capacity of at least 1 wt %, at least 2 wt %, at least 3 wt %, at least 4 wt %, at least 5 wt %, at least 6 wt %, or at least 7 wt %, or from 1 wt % to 14 wt %, or from 4 wt % to 14 wt %. The anionic resin can be a weak or strong anionic resin and can include a functional group and an anionic group. In some embodiments, the functional group includes trimethylamine or a trialkylbenzyl ammonium cation. In some embodiments, the anionic group includes a chlorine or hydroxide anion. The anionic resin can be a macroporous resin with a pore size of from 20 Angstroms to 120 Angstroms (e.g., 40 to 80 Angstroms). In some embodiments, the anionic resin has a particle size or from 16 to 50 mesh size. Exemplary anionic resins include Amberlite IRA-400 having a trialkylbenzyl ammonium functional group and a chloride anion and Siemens A-674 OH having a trimethylamine functional group and a hydroxide anion. In some embodiments, the anionic resin can have a high exchange capacity, e.g., from 1 to 1.4 meq/ml.

In some embodiments, the gas dehydration system and the anionic resin bed 28 can include an adsorbent selected from activated carbon, alumina, silica, zeolite, an iron-oxide-based adsorbent (e.g., SULFATREAT) or a supported metal. This adsorbent can be used in addition to the anionic resin to remove the sulfur-containing additive and/or mercury from the rich glycol stream 16. In some embodiments, an ion exchange resin other than an anionic resin can be used to remove the sulfur-containing additive and/or mercury from the rich glycol stream 16.

In some embodiments, the anionic resin bed 28 can be an in-line resin bed. In some embodiments, the anionic resin bed 28 can be provided as at least two anionic resin beds in parallel thus allowing (e.g., through a series of valves) a first anionic resin bed to be used to remove at least a portion of the sulfur-containing additive and/or the mercury from the rich glycol stream 16 while a second anionic resin bed is regenerating using an alkaline or chloride solution. The second anionic resin bed once it has been regenerated can then be used to remove at least a portion of the sulfur-containing additive and/or the mercury from the rich glycol stream 16 while the first anionic resin bed is regenerated.

In some embodiments, the anionic resin bed 28 can be a single in-line resin bed that is a throwaway bed provided to last for several years. The anionic resin in the throwaway anionic resin bed 28 can be removed when the system is shut down and replaced with fresh anionic resin.

Upon leaving the anionic resin bed 28, the rich glycol stream 16 now including lower amounts of sulfur-containing additive can proceed to a cross exchanger 30 where the rich glycol stream is heated by a lean glycol stream 32. The rich glycol stream 16 can then be fed to a glycol regenerator 34. The glycol regenerator 34 can be in the form of a tower. The top of the glycol regenerator 34 can include a condenser 36 operating between 90 and 110° C. The condenser 36 produces a water vapor stream 40 and can be associated with an adsorbent such as activated carbon as discussed herein to remove mercury vapor and/or an iron-oxide-based adsorbent (e.g., SULFATREAT) to remove hydrogen sulfide. The bottom of the glycol regenerator 34 can include a reboiler 38 operating at between 190 and 205° C. The reboiler can produce the lean glycol stream 32. As noted herein, the sulfur-containing additive can undesirably degrade at the higher temperatures (e.g., the 190 to 205° C.) in the glycol regenerator 34. The degradation products of the sulfur-containing additive can include thiosulfates, bisulfites, sulfites, sulfates, elemental sulfur, hydrogen sulfide, sulfur-hydrocarbon complex or a combination thereof, and these degradation products and the mercury can end up in the lean glycol stream 32. These products were identified after exposing the glycol-polysulfide-mercury stream to 200° C. and detecting the decomposition products using a Mass Spectrometer.

In some embodiments, the mercury can be vented off with the regenerator gas stream 40.

Hydrogen sulfide was found to be a product of decomposition of the sodium polysulfide agent under the elevated operating temperature of the regenerator (200C). The hydrogen sulfide that evolves during the regeneration process can be captured by a separate adsorption bed containing a suitable adsorbent for H2S, such as zinc oxide and iron oxide (Sulfatreat). The bed is to be located at stream 40. If an MRU unit is installed, the preferred bed location is downstream of the MRU, since part of the H2S may be adsorbed by the MRU bed, but not necessarily all the H2S evolved in the regeneration process.

The regenerated or lean glycol stream 32 can be cooled by the cross exchanger 30 and can then recirculate through a pump 42 back to the liquid contactor. As shown in FIG. 1, in addition to or instead of the anionic resin bed 28, the gas dehydration system can include an anionic resin bed 44 to remove at least a portion of the sulfur-containing additive, the degradation product of a sulfur-containing additive, and/or the mercury from the lean glycol stream 32. In some embodiments, the anionic resin bed 44 can reduce the content of the sulfur-containing additive and/or the content of the degradation product of the sulfur-containing additive in the lean glycol stream by at least 10%, at least 20%, at least 30%, at least 40%, at least 50%, at least 60%, at least 70%, at least 80%, or at least 90% by weight. The anionic resin bed 44 can include the anionic resins and optional adsorbent described with respect to anionic resin bed 28 or the anionic resin can be replaced by an ion exchange resin other than an anionic resin. The anionic resin bed 44 can be provided as a throwaway bed or can be provided as at least two anionic resin beds in parallel thus allowing a first anionic resin bed to be used to remove at least a portion of the sulfur-containing additive, the degradation product of a sulfur-containing additive, and/or the mercury from the lean glycol stream 32 while a second anionic resin bed is regenerating using an alkaline or chloride solution.

The lean glycol stream 32 leaving the anionic resin bed 44 becomes the glycol feed 12 when it is recycled back to the glycol contactor 14. As the sulfur-containing additive is removed using the anionic resin bed 28 and/or anionic resin bed 44, additional sulfur-containing additive can be added to the lean glycol stream 32/glycol feed 12 before it is provided to the glycol contactor 14. Additional glycol can also be added to the lean glycol stream 32/glycol feed 12 before it is provided to the glycol contactor 14 to provide at least 97% glycol in the glycol feed 12.

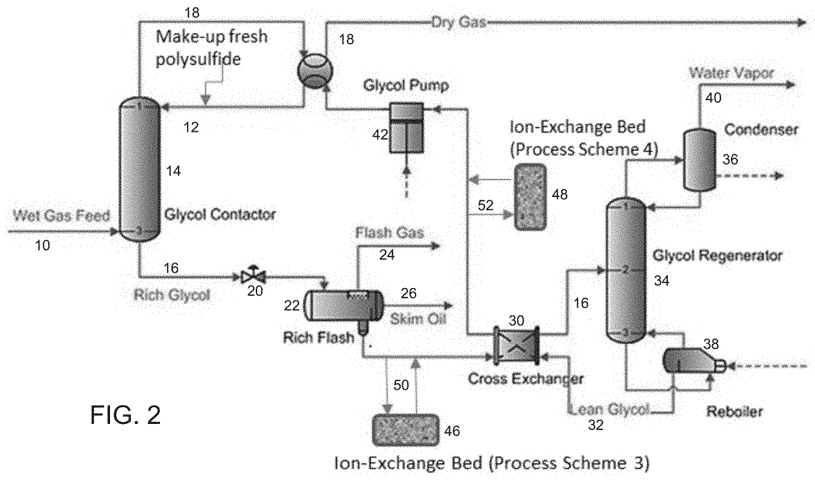

FIG. 2 illustrates an alternative embodiment to FIG. 1 wherein anionic resin bed 46 and anionic resin bed 48 are provided instead of in-line anionic resin bed 28 (provided upstream of the glycol regenerator 34) and anionic resin bed 44 (provided downstream of the glycol regenerator 34). In FIG. 2, a slip stream 50 is diverted from rich glycol stream 16 to anionic resin bed 46 and/or a slip stream 52 is diverted from lean glycol stream 32 to anionic resin bed 48. The anionic resin beds 46 and 48 operate in the manner described above for anionic resin beds 28 and 44, respectively, except that only a portion of the rich glycol stream 16 and lean glycol stream 32 is fed to anionic resin beds 46 and 48. For example, 0.5%-50% by volume (e.g., 0.5%-2% by volume) of the rich glycol stream 16 and/or the lean glycol stream 32 can be fed to anionic resin beds 46 and 48, respectively. Anionic resin beds 46 and 48 can be provided as throwaway beds or can be provided as two or more anionic beds in parallel as discussed above to allow a first anionic resin bed to remove sulfur-containing additive, a degradation product of the sulfur-containing additive and/or mercury from the stream while a second anionic resin bed is regenerating using an alkaline or chloride solution.

### EXAMPLES

The following examples are for the purpose of illustration of the invention only and are not intended to limit the scope of the present invention in any manner whatsoever.

### Example 1

Experiments were run to evaluate the removal of polysulfide (measured as total sulfur) using an anionic resin. The experiments consisted in contacting resins and activated carbons with a solution containing 94 wt % triethylene glycol and 6 wt % water (typical of rich glycol) and a known concentration of sodium polysulfide (Table 1) at room temperature. The experiments were typically run overnight by shaking glass vials vigorously for 24 hours until an equilibrium concentration between the liquid solution and the solids was established. The initial and final total sulfur content was measured via the ICP analytical method and the capacity of the solids for removing sulfur calculated.

TABLE 1
Removal of sulfur using resins and activated carbon from a TEG/water
feed containing 1000 ppm sulfur using 24-hour batch equilibrium tests
at room temperature.

| | | Resin | Feed | S | S |
|---|---|---|---|---|---|
| Feed Used | Resin | (g) | (g) | (ppm) | (wt %) |
| C5260-17 | Purolite, FerrIX A33E | 1.0052 | 200.65 | 838 | 3.0 |
| C5260-17 | Siennens, A-674 OH | 1.0074 | 200.66 | 708 | 5.6 |
| C5260-17 | Carbon WVB-1500 | 1.0441 | 200.37 | 934 | 1.0 |
| C5260-17 | Carbon Nuchar 295-R-03 | 1.0165 | 200.1 | 968 | 0.4 |
| C5260-17 | Amberlite IRA-400 Chloride | 1.0111 | 200.66 | 691 | 5.9 |

### Example 2

Example 2 was conducted in the same manner as Example 1 but using one specific anionic resin under different initial sulfur concentration to look at a wider range of sulfur concentrations.

TABLE 2
Removal of polysulfide using a resin from a TEG/water feed containing
235 to 3632 ppm polysulfide. The loadings are for total sulfur removal
and the results are from 24-hour batch equilibrium tests at room
temperature.

| | | 94% | | | | Total |
|---|---|---|---|---|---|---|
| | | TEG | | S | S | S |
| | Feed | Sol'n | Resin | initial | final | wt |
| Resin | (g) | (g) | (g) | (ppm) | (ppm) | % |
| | 10.29 | | | | | |
| Amberlite IRA-400 Chloride | 100.06 | 0 | 1.02 | 3632 | 2910 | 7.1 |
| Amberlite IRA-400 Chloride | 75.07 | 25.07 | 1.06 | 2658 | 2090 | 5.4 |
| Amberlite IRA-400 Chloride | 50.2 | 50.01 | 1.00 | 1772 | 1120 | 6.5 |
| Amberlite IRA-400 Chloride | 25.1 | 75.13 | 1.14 | 939 | 364 | 5.1 |
| Amberlite IRA-400 Chloride | 12.52 | 87.54 | 1.02 | 469 | 188 | 2.8 |
| Amberlite IRA-400 Chloride | 6.29 | 93.99 | 1.07 | 235 | 96.1 | 1.3 |

### Example 3

Table 3 shows examples of mercury removal using resins (one of them is provided in Example 1). Some of these resins can be used for targeting polysulfide removal and have an extra benefit of simultaneously removing mercury. The experiments were conducted by contacting a solution of 94 wt % triethylene glycol and 6 wt % water (typical of rich glycol) and a known concentration of sodium polysulfide at room temperature.

TABLE 3
24-hour batch equilibrium tests for mercury removal at room
temperature. Polysulfide measured as total sulfur was 400 ppm.

| | Solution, | Hg Loading |
|---|---|---|
| Resin | Hg (ppb) | (wt %) |
| None | 2720 | 0 |
| Purolite A103 | 2530 | 0.1 |
| Plus | | |
| Purolite A111 | 2503 | 0.1 |
| Purolite MN100 | 2573 | 0.1 |
| Siemens A-674 | 2530 | 0.1 |
| OH | | |

TABLE 4
below shows a comparison between commercial resins with high affinity
for mercury and Siemens A-674 OH, which is not marketed for mercury
removal. Commercial resins showed up to one order of magnitude less
loading for mercury than our best performing resin.

| | Initial Hg | Hg | |
|---|---|---|---|
| | concentration | loading | Feed:Adsorbent |
| Resin | (ppb) | (wt %) | Ratio (g:g) |
| None | 848 | 0 | 100:1 |
| Ambersep GT-74 | 848 | 0.02 | 100:1 |
| Siemens A-674 OH | 848 | 0.1 | 100:1 |
| Lewatit TP-214 | 848 | 0.01 | 100:1 |

Ambersep GT-74 (Lenntech) has been developed for the removal of mercury and is commercialized by Lenntech. Lenntech has stated that the selectivity sequence is the highest for mercury, as follows:

Hg>Ag>Cu>Pb>Cd>Ni>Co>Fe>Ca>Na.

Lewatit® MonoPlus TP 214 is a chelating resin having a high affinity for mercury. It is commercialized by Lanxess. The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A process for removing a sulfur-containing additive or a degradation product thereof in a gas dehydration system, comprising contacting a stream comprising the sulfur-containing additive or a degradation product thereof with an anionic resin to remove at least a portion of the sulfur-containing additive or a degradation product thereof from the stream wherein the gas dehydration system includes a glycol regenerator, and the stream is contacted with the resin, downstream of the glycol regenerator.

2. The process according to claim 1, wherein the stream further comprises mercury and the anionic resin removes at least a portion of the mercury from the stream.

3. The process according to claim 1 or 2, wherein the anionic resin is contacted with an alkaline or chloride solution to regenerate the anionic resin after the anionic resin has been used to remove the sulfur-containing additive or the degradation product thereof.

4. The process according to claim 3 or 4, wherein the gas dehydration system includes at least two anionic resin beds and wherein the stream can contact a first anionic resin bed while a second resin bed is regenerating.

5. The process according to any one of claims 1-5, wherein the process includes a sulfur-containing additive and the sulfur-containing additive is selected from the group consisting of sulfides, hydrosulfides, inorganic and organic polysulfides, inorganic and organic thiocarbamates, mercaptans, thiourea, sulfur-containing polymers, sulfanes, and combinations thereof.

6. The process according to claim 6, wherein the process the sulfur-containing additive comprises a sulfide selected from the group consisting of a polysulfide, a polysulfide salt, a sulfide salt, or a combination thereof.

7. The process according to claim 6, wherein the sulfide comprises sodium polysulfide.

8. The process according to any one of claims 1-7, wherein the process includes removing a degradation product of a sulfur-containing additive, wherein the polysulfide degradation product comprises a thiosulfate, a sulfite, a sulfate, elemental sulfur, hydrogen sulfide, or a combination thereof.

9. The process according to one of claims 1-8, wherein the anionic resin has a sulfur capacity of at least 1 wt %, at least 2 wt %, at least 3 wt %, at least 4 wt %, at least 5 wt %, at least 6 wt %, or at least 7 wt %, or from 1 wt % to 14 wt %, or from 4 wt % to 14 wt %.

10. The process according to any one of claims 1-9, wherein the anionic resin has a pore size of from 20 Angstroms to 120 Angstroms.

11. The process according to any one of claims 1-10, wherein the stream comprises at least 94 wt % glycol.

12. The process according to any one of claims 1-11, wherein the glycol comprises triethylene glycol.

13. The process according to claim 5, wherein the content of the sulfur-containing additive in the stream is reduced by at least 50%, at least 60%, at least 70%, at least 80%, or at least 90%.

14. The process according to claim 1 or 8, wherein the content of the sulfur-containing additive and/or the degradation product of the sulfur-containing additive in the stream is reduced by at least 50%, at least 60%, at least 70%, at least 80%, or at least 90%.

15. The process according to any one of claims 1-14, wherein the anionic resin includes a chlorine or hydroxide anion.

16. The process according to any one of claims 1-15, wherein the anionic resin includes a trimethylamine or trialkylbenzyl ammonium functional group.

17. The process according to any one of claims 1-16, wherein an adsorbent selected from activated carbon, alumina, silica, zeolite, iron-oxide based adsorbent, or a supported metal is used in addition to the anionic resin to remove the sulfur-containing additive, the degradation product of a sulfur-containing additive, and/or the mercury from the stream.

18. The process according to claim 17, wherein the resin comprises an iron-oxide-based adsorbent that removes at least a portion of hydrogen sulfide from the stream.
